(19) United States
(12) Patent Application Publication
(10) Pub. No.: US 2009/0254058 A1
(43) Pub. Date: Oct. 8, 2009

(54) Absorbent Article with Registration Mark

(76) Inventors: Makoto Shiriike, Kobe (JP); Seiji Tsuneki, Akashi (JP); Seiichi Ohide, Kakogawa (JP); Hiroyuki Ueno, Kobe (JP)

Correspondence Address: THE PROCTER & GAMBLE COMPANY;Global Legal Department - IP, Sycamore Building - 4th Floor, 299 East Sixth Street, CINCINNATI, OH, 45202, US

(21) Appl. No.: 12/419,539

(22) Filed: Apr. 7, 2009

Related U.S. Application Data
(60) Provisional application No. 61/123,309, filed on Apr. 7, 2008.

(51) Int. Cl. A61F 13/84

(52) U.S. Cl. 604/385.01

(57) ABSTRACT

An absorbent article comprising a backsheet, comprising a graphic and the registration mark are located on opposite surfaces to each other. The backsheet extends in a first direction and in a second direction perpendicular to the first direction. The backsheet has a first surface, a second surface opposing to the first surface, a first edge and a second edge each extending substantially in the first direction, and a third edge and a fourth edge each extending substantially in the second direction. The backsheet is provided with a graphic and a registration mark for adjusting the graphic to a predetermined position in the absorbent article. The registration mark is detected when the absorbent article is conveyed in the first direction in the manufacturing process. The graphic is provided on the first surface. The graphic has a first direction component and a second direction component. The second direction component extends substantially continuously from the first edge to the second edge. The registration mark being provided on the second surface.

### CROSS REFERENCE TO RELATED APPLICATION

This application claims the benefit of U.S. Provisional Application No. 61/123,309, filed Apr. 7, 2008.

### FIELD OF THE INVENTION

The present invention relates to an absorbent article comprising a graphic on a first surface of a backsheet and a registration mark on a second surface of the backsheet.

### BACKGROUND OF THE INVENTION

Nowadays, many absorbent articles comprise graphics to enhance their aesthetic appearance and their consumer acceptance. Such graphics are usually preprinted on a part of a layer positioned away from the wearer's body during use (often called “backsheet”) such that the wearer or the caretaker can see the graphics better when the absorbent article is worn.

For absorbent articles, e.g., disposable diapers, it is often desired to adjust the graphic to a predetermined position in the absorbent article. For example, when the absorbent article comprises a large-sized character picture as a graphic, the character picture looks better when the whole character picture is located in the front region of the absorbent article or in the rear region thereof than when it is located in the crotch region thereof or when a portion of the character picture is missing. Also, when the absorbent article comprises a garment-like graphic, the garment-like graphic should be located at a correct position in the absorbent article. For example, in a garment-like graphic comprising a portion simulating a belt, the simulated belt looks better when it is located in the waist region of the absorbent article than otherwise. In these cases, it is desirable to provide the graphic to be correctly located in the absorbent article.

In the manufacturing process of absorbent articles, the detection of the registration mark serves to adjust the graphic to a predetermined position in the absorbent article. A backsheet on which a graphic is printed is fed from a web roll. Based on the detection of the registration mark, the graphic on the backsheet is adjusted to a predetermined position in the absorbent article and attached to other components of the absorbent article (e.g., topsheet, absorbent core, and the like). The registration mark often adopts a color for detection. The color of the registration mark is usually differentiated from the color(s) used for the graphic to avoid detection error.

In order to attract consumers, the graphic can occupy a large part of the backsheet. In such a case, when the graphic and the registration mark are overlapped to each other in the backsheet, the registration mark may not be detected correctly. When the graphic and the registration mark comprise the same color, the graphic may be mistakenly detected as a registration mark. That is, the relationship between the registration mark and the graphic may sometimes cause a limitation on color, size and/or position.

The problem becomes more serious when a series of absorbent articles manufactured in one manufacturing line comprises different graphics and colors from one another. When the backsheet comprises a plurality of graphics, the choice of the color used for the registration mark is limited.

Furthermore, when the registration mark is perceivable from the external side of the absorbent article, the consumers may wonder why such a mark exists when it does not serve to enhance the aesthetic appearance.

There has been a need for an absorbent article comprising a graphic and a registration mark, wherein there is no limitation in the choice of color, size and/or position of the graphic and the registration mark. There has been also a need of an absorbent article providing good aesthetic appearance.

Thus, one of the purposes of the present invention is to provide an absorbent article having a graphic and a registration mark in the backsheet, and the graphic and the registration mark do not interfere with each other in the same surface of the backsheet.

### SUMMARY OF THE INVENTION

The present invention relates to an absorbent article comprising a backsheet, which comprises a graphic and the registration mark located on opposite surfaces to each other. The backsheet extends in a first direction and in a second direction perpendicular to the first direction. The backsheet has a first surface, a second surface opposing to the first surface, a first edge and a second edge each extending substantially in the first direction, and a third edge and a fourth edge each extending substantially in the second direction. The backsheet is provided with a graphic and a registration mark for adjusting the graphic to a predetermined position in the absorbent article. The registration mark is detected when the absorbent article is conveyed in the first direction in the manufacturing process. The graphic is provided on the first surface. The graphic has a first direction component and a second direction component. The second direction component extends substantially continuously from the first edge to the second edge. The registration mark being provided on the second surface.

In one aspect of the invention, the backsheet comprises a single graphic, and the second direction component of the single graphic extends substantially continuously from the first edge to the second edge.

In another aspect of the invention, the backsheet comprises a plurality of graphics and each of the second direction components of the plurality of graphics extends only in a portion between the first edge and the second edge. The plurality of the second direction components forms a composite second direction component, wherein the composite second direction component extends substantially continuously from the first edge to the second edge.

The foregoing answers the need for an absorbent article that can obviate the detection error in the manufacturing process of the absorbent article and thus allows the manufacturer to provide a graphic having arbitrary color, size and/or position regardless of the color, size and/or position of the registration mark.

These and other features, aspects, and advantages of the present invention will become evident to those skilled in the art from reading of the present disclosure.

### BRIEF DESCRIPTION OF THE DRAWINGS DRAWINGS

FIG. 1 is a plan view of a disposable pull-on diaper in a flat and uncontracted state wherein a part of the diaper is cut away for illustration.

FIG. 2 is a simplified plan view of the backsheet used for the diaper of FIG. 1 emphasizing the relationship between the registration mark and the graphic.

FIG. 3 is a simplified plan view of a backsheet comprising a plurality of graphics.

FIG. 4 is another simplified plan view of a backsheet comprising a graphic.

FIG. 5 is a magnified view of the backsheet of FIG. 4 surrounded by a circle 5.

FIG. 6 is a cross-sectional view of the backsheet of FIG. 5 taken along by 6-6′ line.

FIG. 7 is a top plan view of a strip of a backsheet web comprising a repeating disposed backsheet.

FIG. 8 is a top plan view of a strip of a backsheet web comprising a repeating disposed backsheet.

### DETAILED DESCRIPTION OF THE INVENTION

All references cited herein are incorporated herein by reference in their entireties. Citation of any reference is not an admission regarding any determination as to its availability as prior art to the claimed invention.

As used herein, the term “comprising” means that other steps and other ingredients which do not affect the end result can be added. This term encompasses the terms “consisting of” and “consisting essentially of.”

As used herein, the term “absorbent article” refers to devices which absorb and contain body exudates, and more specifically, refers to devices which are placed against or in proximity to the body of the wearer to absorb and contain the various exudates discharged from the body. “Absorbent article” is intended to include diapers, training pants, sanitary napkins, pantiliners, incontinence pads, sweat-absorbent underarm pads, nursing pads, adult incontinence diapers, human waste management devices and the like.

The term “disposable” is used herein to describe absorbent articles which are not intended to be laundered or otherwise restored or reused as an absorbent article (i.e., they are intended to be discarded after a single use, and preferably, to be recycled, composted or otherwise disposed of in an environmentally compatible manner).

The term “diaper” refers to an absorbent article generally worn by infants and incontinent persons that is worn about the lower torso of the wearer. Diapers include, for example, “a conventional diaper” and “a pull-on diaper.”

The term “conventional diaper” herein refers to a diaper which comprises closure means so as to form the diaper into an essentially closed configuration around the wearer's body (e.g. taped diaper). The term “pull-on diaper” herein refers to a diaper which have a defined waist opening and a pair of leg openings and which are pulled onto the body of the wearer by inserting the legs into the leg openings and pulling the article up over the waist.

The term “body-facing surface” herein refers to a surface of an absorbent article and/or its component member which faces the body of the wearer. The term “external surface” herein refers to the opposite surface to the body-facing surface that faces away from the wearer when the absorbent article is worn. The absorbent article and a composite thereof (i.e., the topsheet, the absorbent core and the backsheet, or any member thereof) comprises a body-facing surface and an external surface opposing to the body-facing surface.

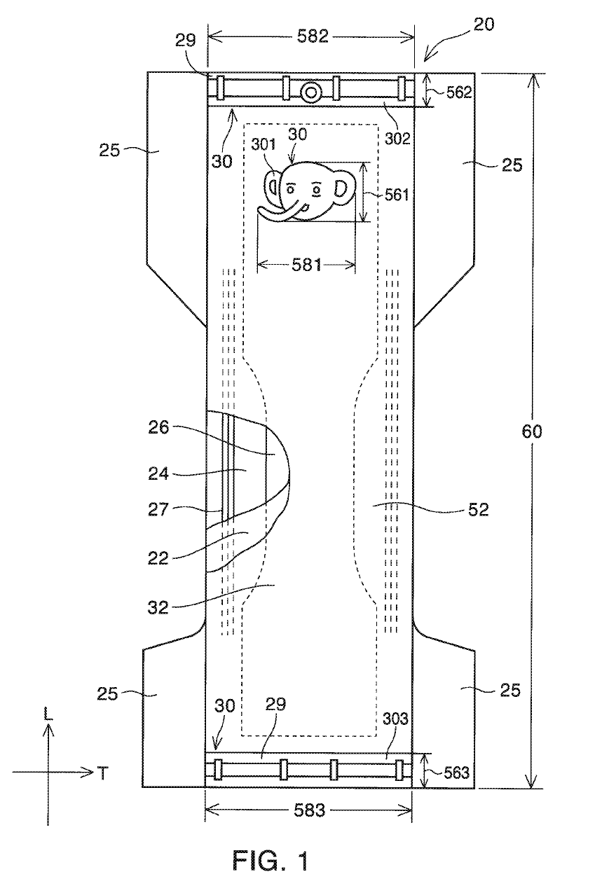

FIG. 1 illustrates a top plan view of a disposable pull-on diaper which is representative of a disposable absorbent article, in a flat and uncontracted state. A part of the diaper is cut away for illustration. The diaper 20 generally comprises a backsheet 22, a topsheet 24 joined with the backsheet 22 and an absorbent core 26 between the backsheet 22 and the topsheet 24. The topsheet 24 is located to face the body or nearest the body when the diaper 20 is worn and is generally provided with a liquid permeable region so that body exudates can flow through the topsheet 24 to the absorbent core 26. The backsheet 22, which is placed away from the body during wear, may be liquid impermeable so that outer clothing or other articles are not wetted by the body exudates. The backsheet 22 may comprise a polymeric film (hereinafter referred to as “backsheet film”). The body-facing surface of the backsheet 22 may be attached to the absorbent core 26 and the topsheet 24 by a conventional attaching means. The external surface of the backsheet 22 may further be laminated to a layer of nonwoven 32 by a conventional attaching means, in which case there is provided a more cloth-like and garment-like feel than is obtained with a polymeric film backsheet only.

The diaper 20 has elastically extensible side panels 25 provided to ensure more comfortable and contouring fit by initially conformably fitting the pull-on diaper 20 to the wearer and sustaining this fit throughout the time of wear well past when it has been loaded with exudates. Leg elastics 27 and a waist elastic 29 are also provided to enhance the fit around the legs and the waist, respectively. The side panels 25 are joined at seams to form a waist opening and leg openings with the pull-on diaper 20.

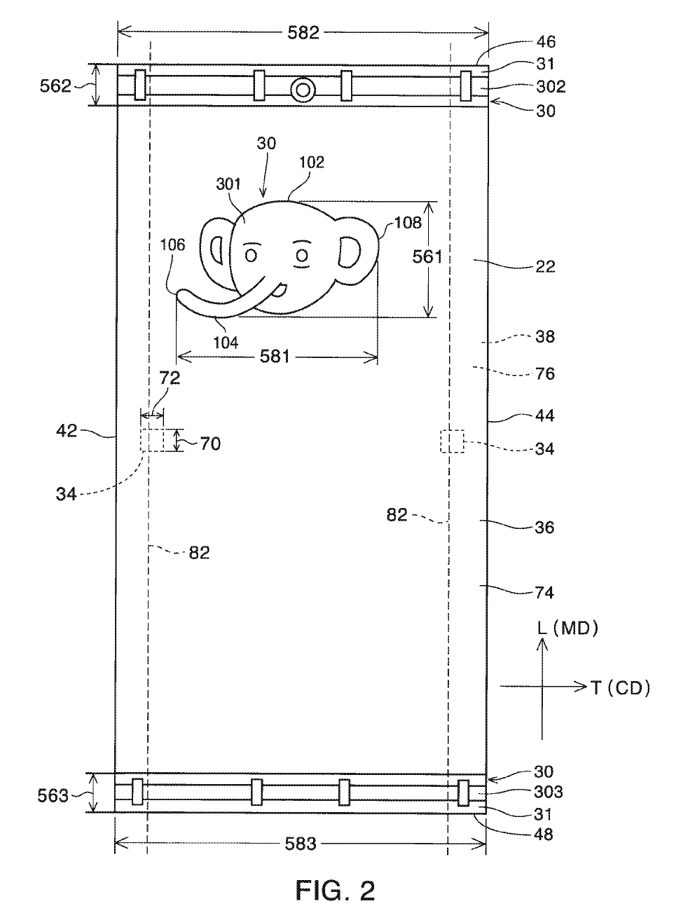

FIG. 2 illustrates a simplified plan view of the backsheet of the disposable pull-on diaper of FIG. 1. For the purpose of illustration, the ratio of the length and the width is modified. The backsheet 22 extends in a first direction and in a second direction perpendicular to the first direction. The backsheet 22 extends in a longitudinal direction L and in a transverse direction T perpendicular to the longitudinal direction L. The backsheet 22 also has a thickness direction (not shown) perpendicular to the longitudinal direction L and the transverse direction T. In this embodiment, the first direction is the longitudinal direction L, and the second direction is the transverse direction T. In another embodiment, the first direction may be the transverse direction T and the second direction may be the longitudinal direction L. The backsheet 22 comprises a first surface 36 and a second surface 38 opposing to the first surface 36. In the embodiment of FIG. 2, the first surface 36 is the external surface 74 and the second surface 38 is the body-facing surface 76. In another embodiment, the first surface 36 may be the body-facing surface and the second surface 38 may be the external surface.

The backsheet 22 has a first edge and a second edge each extending substantially in the first direction, and a third edge and a fourth edge each extending substantially in the second direction. In the embodiment of FIG. 2, the first edge is a first longitudinal edge 42 and the second edge is a second longitudinal edge 44. The first longitudinal edge 42 and the second longitudinal edge 44 each extends substantially in the longitudinal direction L. At the same time, the third edge is a first transverse edge 46 and the fourth edge is a second transverse edge 48. The first transverse edge 46 and the second transverse edge 48 each extends substantially in the transverse direction T. In another embodiment, the first edge may be a first transverse edge 46, and the second edge may be a second transverse edge 48, where the first transverse edge and the second transverse edge may each extend substantially in the transverse direction T. The third edge may be a first longitudinal edge 42 and the fourth edge may be a second longitudinal edge 44, where the first longitudinal edge and the second longitudinal edge may each extend substantially in the longitudinal direction L.

The backsheet 22 is provided with a graphic 30 on the first surface 36. In the embodiment of FIGS. 1 and 2, the graphic 30 comprises a first graphic 301 of an elephant, a second graphic 302 and a third graphic 303 both of which are evocative of a waist band in the waist region 31. The graphics 301, 302 and 303 (collectively referred to as the “graphic 30”) are printed on the external surface 74 of the backsheet 22. The graphic 30 can be printed by any conventional printing methods or technologies known in the art, including, but not limited to, a gravure printing, a flexo printing, an offset printing, an ink jet printing, and the like.

In the embodiment of FIG. 2, the first longitudinal edge 42 is a left edge of the backsheet 22, and the second longitudinal edge 44 is a right edge of the backsheet 22, the first transverse edge 46 is an upper edge of the backsheet 22 and the second transverse edge 48 is a lower edge of the backsheet 22.

Each of the graphics of the present invention comprises a first direction component and a second direction component. In the embodiment of FIG. 2, the first graphic 301 comprises a first longitudinal component 561 and a first transverse component 581. The first longitudinal component 561 is a portion extending longitudinally from an uppermost periphery 102 of the first graphic 301 to a lowermost periphery 104 of the first graphic 301. The first transverse component 581 is a portion extending transversely from a leftmost periphery 106 of the first graphic 301 to a rightmost periphery 108 of the first graphic 301. Similarly, the second graphic 302 comprises a second longitudinal component 562 and a second transverse component 582, and the third graphic 303 comprises a third longitudinal component 563 and a third transverse component 583. In the embodiment of FIG. 2, the first direction component is a longitudinal component and the second direction component is a transverse component. In another embodiment, the first direction component may be a transverse component and the second direction component may be a longitudinal component.

The term “graphic” may refer, but is not limited, to an image, a design, a pattern, symbology, indicia, or the like. “Graphic” includes, for example, animals (e.g., dogs, cats, bears, squirrels, tigers, lions, mice, foxes, and the like); birds (e.g., swallows, sparrows, hawks, ducks, eagles, swans, and the like); human beings; plants such as flowers (e.g., dandelions, roses, tulips, cherry blossoms, sunflowers, carnations, and the like), trees and leaves; stars; moons; cartoon characters; toys (e.g., dolls, bats, balls, rackets, and the like); electric instruments (e.g., mobile phones, computers, and the like); ornaments (e.g., frills, ribbons, buttons, belts, neckties, caps, hats, and the like); garment patterns (e.g., denims, checks, frills, and the like); seasonal things or goods such as a snowman; landscapes (e.g., trees, mountains, sun, moon, oceans, grass, and the like); geometrical patterns (e.g., lines, stripes, circles, ovals, squares, triangles, waves, and the like); literal characters (e.g., alphabets, numerals, Chinese characters, Japanese characters, and the like), and the like. “Graphic” also includes a uniformly colored pattern and a variously colored pattern which occupy a certain area within the absorbent article. The term “colored” may refer to a status of non-white.

The backsheet 22 is provided with a registration mark 34 on the second surface 38. The term “registration mark” herein refers to a mark which is used for adjusting the graphic of the backsheet to a predetermined position in the absorbent article. Registration marks are used so that machinery such as detectors of the combining and cutting mechanisms can detect the registration marks to properly adjust the backsheet or the combination of the backsheet and the other components (e.g., topsheet, absorbent core, nonwoven film, and the like), and to adjust the graphic to a predetermined position of each diaper without significant variation. Therefore, it is desired to correctly detect the position of the registration mark in the backsheet.

In the embodiment of FIG. 2, the backsheet 22 comprises a pair of registration marks 34 on the second surface 38. The first surface 36 comprising the graphic 30 is the external surface 74 and the second surface 38 comprising the registration marks 34 is the body-facing surface 76 for aesthetic acceptance of consumers. Otherwise, the first surface 36 comprising the graphic 30 may be the body-facing surface 76 and the second surface 38 comprising the registration marks 34 may be the external surface 74.

The registration mark may be a color, a pattern, or anything else which can be detected by detectors (e.g., CCD cameras, ultraviolet detectors or infrared detectors). The registration mark may provide an optical marker which operates on the basis of providing detectable changes in the intensities of visible and/or non-visible wavelengths of light. The registration mark may be characterized by a single color such as yellow, blue, red, green, light green, light blue, orange, pink, gray, black, beige, brown, light brown, and the like. In the embodiment of FIG. 2, the registration mark 34 is a “yellow” color. The registration mark 34 may be configured in any desired size or shape. In the representatively shown construction, for example, the registration mark 34 may comprise a generally square area which has a longitudinal direction length 70 of about 1 centimeter and a transverse width 72 of about 1 centimeter. Of course, other lengths and widths may be employed depending on the sensitivity or, performance of the detector.

Referring to FIG. 2, the registration mark 34 is detected by a detector when the diaper 20 (also the backsheet 22) is conveyed in the first direction in the manufacturing process. The backsheet 22 comprises a first direction axis 82 extending in the first direction. The first direction axis 82 represents a detector pathway on the backsheet 22 in the manufacturing process to detect the registration mark 34. The first direction axis 82 may be located on the first surface 36 and/or the second surface 38 of the backsheet 22.

The backsheet 22 is conveyed in a machine direction MD for assembling the absorbent article in the manufacturing process. In the embodiment of FIG. 2, the machine direction MD corresponds to the longitudinal direction L of the backsheet 22. The cross machine direction CD is perpendicular to the machine direction MD as illustrated in FIG. 2. The cross machine direction CD corresponds to the transverse direction T of the backsheet 22. As the backsheet 22 is conveyed in the machine direction MD in the manufacturing process, the detector runs on the backsheet 22 along the first direction axis 82. After the detector detects the registration mark 34, the detector transmits positional information to machinery for registering the backsheet 22 with the other components (e.g., topsheet, absorbent core, and the like). The machinery for registering the backsheet 22 receives the positional information for registering the backsheet 22 and registers the backsheet 22 with the other components. Finally, the graphic 30 provided on the backsheet 22 is correctly adjusted to a predetermined position in the diaper 20.

In the present invention, the second direction component of the graphic 30 extends substantially continuously from the first edge to the second edge. The term “substantially continuously” includes not only the embodiment wherein the second direction component extends completely continuously from the first edge to the second edge, but also the embodiment wherein the second direction component extends almost all (e.g., more than about 80% or more than about 90%) of the distance from the first edge to the second edge. In the embodiment of FIG. 2, the second direction component comprises the first transverse components 581, the second transverse components 582 and the third transverse components 583. The second transverse component 582 of a single graphic 302 substantially continuously extends from the first longitudinal edge 42 to the second longitudinal edge 44. The third transverse component 583 of a single graphic 303 also substantially continuously extends from the first longitudinal edge 42 to the second longitudinal edge 44.

If both the graphic 30 and the registration mark 34 are located on the same surface of the backsheet 22, and the first direction axis 82 runs across not only the registration mark 34 but also the graphic 30, the detector may mistakenly detect the graphic 30 as a registration mark. Such detection error happens, for example, when the color of the registration mark 34 is identical with or similar to the color of the graphic 30 because the detector sometimes cannot distinguish the color difference of the registration mark 34 from the graphic 30. For example, if the registration mark 34 has a “yellow” color, and if the graphic 302 and/or the graphic 303 has a yellow color at a portion crossing the first direction axis 82, the detector may not be able to detect the registration mark correctly because of the color identicalness. This problem of detection error can happen when the graphic and the registration mark have similar hues, even if they do not have an identical color.

Thus, freedom in the choice of color may be limited when the graphic 30 and the registration mark 34 are located on the same surface and the first direction axis 82 runs across the graphic 30 and the registration mark 34. It is troublesome for the manufacturer to change the color of the registration mark 34 depending on the color of the graphic 30. It is also troublesome for the manufacturer to consider changing the color of the graphic 30 depending on the color of the registration mark 34.

In the present invention, even if the second direction component of the graphics 302 and 303 on the first surface 36 of the backsheet 22 extend substantially continuously from the first longitudinal edge 42 to the second longitudinal edge 44, the detection error can be obviated. As described above, the graphic 302 and the graphic 303 are located on the first surface 36 and the registration mark 34 is located on the second surface 38. Accordingly, the detector can detect the registration mark 34 without mistakenly detecting the graphic 302 and/or the graphic 303. The present invention has overcome the design limitation of the graphic 30 and the registration mark 34 (e.g., color, size and position) by locating the graphic 30 and the registration mark 34 on the opposite surfaces to each other. As a result, the manufacturer can design the graphic 30 of the backsheet 22 regardless of color, size and position of the registration marks 34. Similarly, the manufacturer can provide the registration mark 34 of the backsheet 22 regardless of color, size and position of the graphic 22.

The registration mark 34 may be substantially unperceivable from the external side of the absorbent article 20. When the registration mark 34 is provided on the body-facing surface 76 of the backsheet 22, the registration mark 34 is less perceivable than it is located on the external surface 74 of the backsheet 22. Depending on the thickness and the material of the backsheet 22, and also depending on the color of the registration mark 34, the registration mark 34 may not be perceivable from the external side of the absorbent article 20. When the registration mark 34 is substantially unperceivable from the external side of the absorbent article 20, the consumer hardly notices the presence of the registration mark 34 during its normal use. In this embodiment, the presence of the registration mark 34 does not influence the appearance of the graphic 30 in the color, size and the position viewed from the external surface of the absorbent article 20.

Otherwise, the registration mark 34 may be substantially perceivable from the external side of the diaper 20. Depending on the thickness and the material of the backsheet 22, and also depending on the color of the registration mark 34, the registration mark 34 on the body-facing surface 76 of the backsheet 22 may be substantially perceivable from the external side of the diaper 20. Even if the registration mark 34 is substantially perceivable from the external side of the diaper 20, the registration mark 34 may be less perceivable than the graphic 30 on the external surface 74 of the backsheet 22. When the registration mark 34 is substantially perceivable from the external side of the diaper 20, the registration mark 34 may be a part of the graphic 30 to provide an aesthetic effect with the diaper 20.

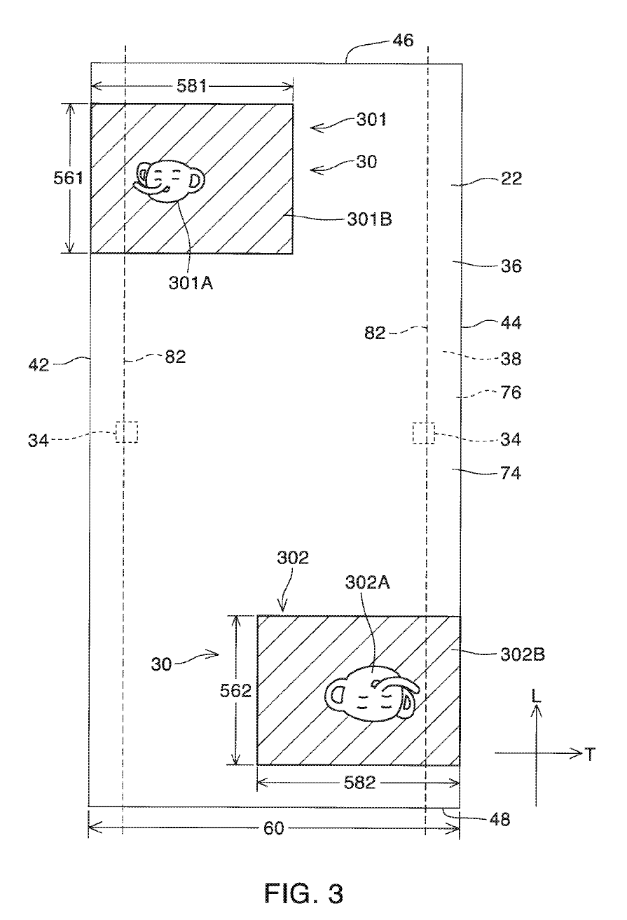

FIG. 3 illustrates a simplified view of another backsheet used for the absorbent article of the present invention. For the purpose of illustration, the ratio of the length and the width of the backsheet is modified. The backsheet 22 is provided with a plurality of graphics 30 on the first surface 36. The plurality of graphics 30 comprises a first graphic 301 and a second graphic 302. The first graphic 301 comprises a graphic 301A of an elephant and a graphic 301B of a colored pattern background. The second graphic 302 comprises a graphic 302A of an elephant and a graphic 302B of a colored pattern background. The graphic 301A is located against the graphic 301B, and the graphic 302A is located against the graphic 302B.

The first graphic 301 comprises a first longitudinal component 561 and a first transverse component 581. The second graphic 302 comprises a second longitudinal component 562 and a second transverse component 582. The first transverse component 581 and the second transverse component 582 form a composite transverse component 60. The “composite” component refers to an assembly of a plurality of components having the same direction. The composite component includes a composite first direction component and a composite second direction component. In an embodiment, the composite first direction may be a composite transverse component and the composite second direction component may be a composite longitudinal component. In another embodiment, the composite first direction may be a composite longitudinal component and the composite second direction component may be a composite transverse component.

The composite second direction component of the graphic extends substantially continuously from the first edge to the second edge. In the embodiment of FIG. 3, the composite second direction component is the composite transverse component 60. The composite transverse component 60 substantially extends from the first longitudinal edge 42 to the second longitudinal edge 44, while each of the transverse components 581 and 582 extends only in a portion between the first edge and the second edge. The backsheet 22 is provided with a pair of registration marks 34 on the second surface 38. The first surface 36 comprising the plurality of graphics 30 is the external surface 74 and the second surface 38 comprising the pair of registration marks 34 is the body-facing surface 76 for aesthetic acceptance of consumers.

In the embodiment of FIG. 3, the first direction axis 82 runs across not only the registration mark 34 but also either the first graphic 301 or the second graphic 302. As described above, the first graphic 301 and the second graphic 302 are located on the first surface 36 and the registration mark 34 is located on the second surface 38. Similar to the embodiment of FIG. 2, the detector can detect the registration mark 34 without mistakenly detect the first graphic 301 and/or the second graphic 302. As a result, the manufacturer can design the graphic 30 of the backsheet 22 regardless of the color, the size and the position of the registration mark 34. Similarly, the manufacturer can provide the registration mark 34 of the backsheet 22 regardless of color, size and position of the graphic 22.

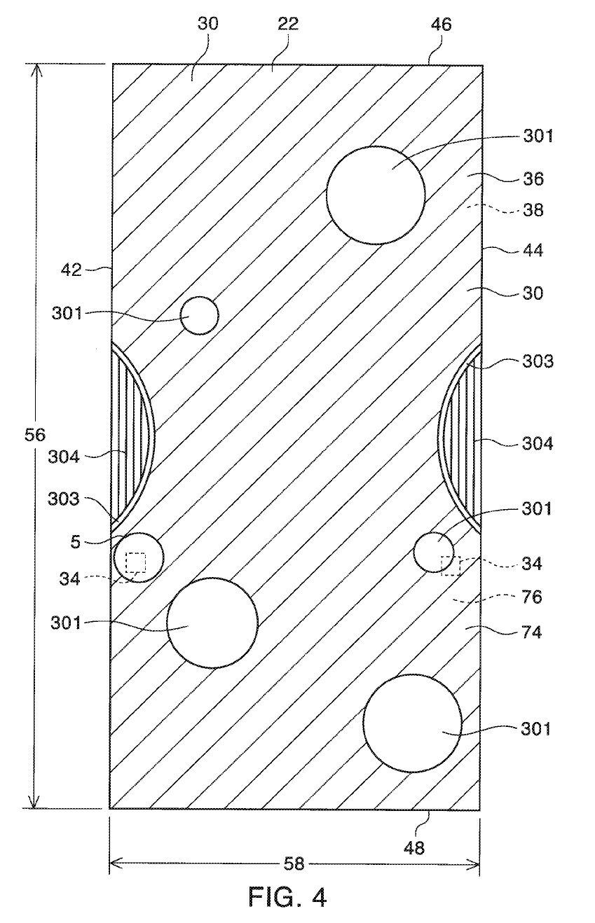

FIG. 4 illustrates another embodiment of the backsheet 22. For the purpose of illustration, the ratio of the length and the width of the backsheet is modified. The backsheet 22 is provided with a graphic 30 on the first surface 36. The graphic 30 comprises a plurality of graphics 301 of polka dots and a graphic 302 of a colored pattern background. The graphics 301 is located against the graphic 302. The graphic 30 also comprises a graphic 303 evocative of a leg elastic band. The graphic 30 also comprises a graphic 304 of a colored pattern which is transversely outside the graphic 303. As a whole, the graphic 30 occupies substantially all of the backsheet 22. “Substantially all of the backsheet” includes, but not limited to, more than about 80% or more than about 90% of the total area of the backsheet 22. The graphic 30 comprises a longitudinal component 56 and a transverse component 58. The backsheet 22 is provided with a registration mark 34 on the second surface 38. In the embodiment of FIG. 4, the first surface 36 comprising the graphic 30 is an external surface 74 and the second surface 38 comprising the registration mark 34 is a body-facing surface 76.

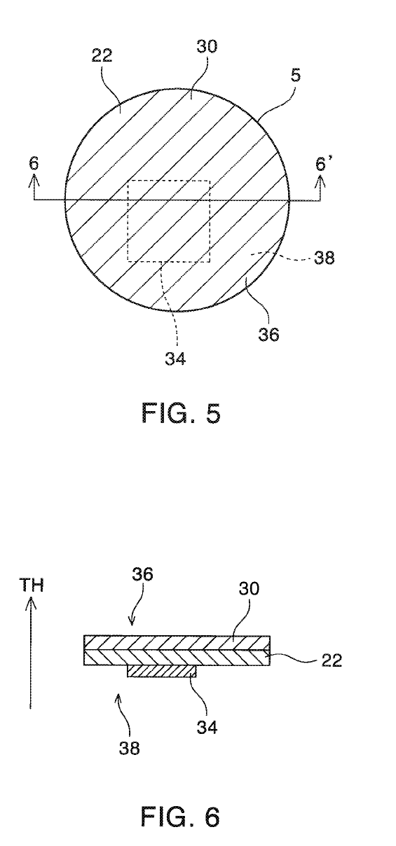

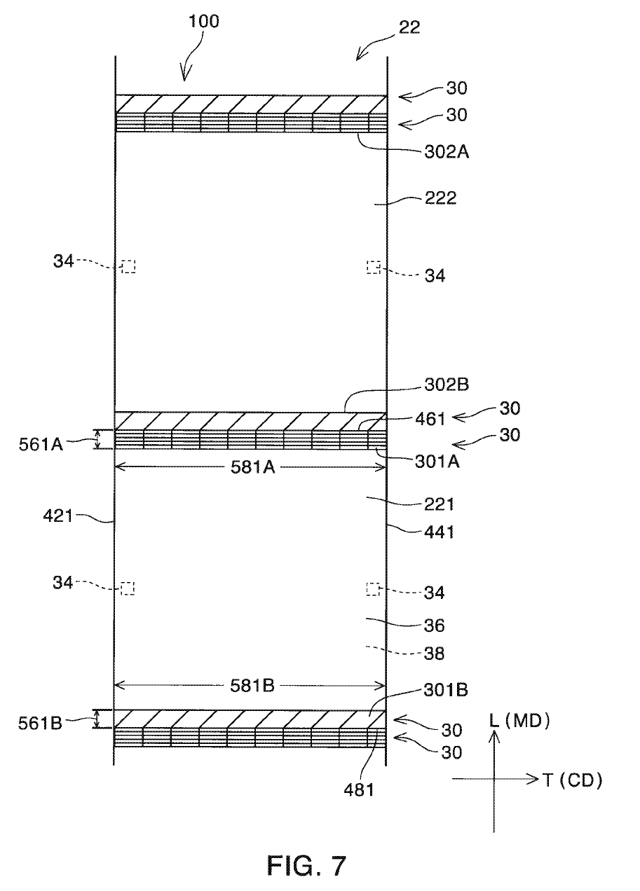

The graphic on the first surface may at least partially cover the registration mark on the second surface in the thickness direction TH (see FIGS. 5 and 6). In the embodiment of FIG. 6, the graphic on the first surface 36 may cover the entirety of the registration mark 34 on the second surface 38 in the thickness direction. Owing to this, the registration mark 34 on the second surface 38 is less perceivable from the side of the first surface 36. When the first surface 36 is an external surface 74, the registration mark 34 is less perceivable from the external side of the absorbent article 20. In the embodiment of FIG. 4, the graphic 30 on the first surface 36 covers the entirety of the registration mark 34 in the thickness direction TH. As the graphic 30 entirely covers the registration mark 34 in the thickness direction TH of the backsheet 22, when the first surface 36 comprising the graphic 30 is an external surface 74 of the backsheet 22, the consumers do not notice the registration mark 34. Alternatively or additionally, the graphic 30 on the first surface 36 may cover a part of the registration mark 34 in the thickness direction TH. As the graphic 30 partially covers the registration mark 34 in the thickness direction TH of the backsheet 22, when the first surface 36 comprising the graphic 30 is an external surface 74 of the backsheet 22, it is less likely that the consumers notice the registration mark 34. Although a part of the registration mark 34 on the body-facing surface 74 may be perceivable from the external surface 76, the registration mark 34 is still less perceivable owing to the concealing effect by the graphic 30.

In the embodiment of FIG. 4, the graphic 30 is provided to occupy substantially all of the backsheet 22. Even in such a case, as the graphic 30 and the registration mark 34 are located on the opposite surfaces to each other, the registration mark 34 is properly detected by a detector without mistakenly detecting the graphic 30 in the manufacturing process. The present invention obviates the detection error of the registration mark even if a large graphic occupying substantially all of the backsheet is provided.

FIG. 7 illustrates a schematic diagram showing a portion of a continuous backsheet web 100 having consecutively spaced graphics and registration mark printed thereon. For the purpose of illustration, the ratio of the length and the width of the backsheet is modified. The backsheet web 100 of FIG. 7 is a status before being attached to the other components (e.g., topsheet, absorbent core, and the like). The backsheet web 100 consists of a series of backsheet 22 to be severed when the absorbent article is assembled. The series of backsheet 22 is provided with a graphic 30 respectively. For example, the backsheet web 100 comprises a first backsheet 221 and a second backsheet 222. The first backsheet 221 is provided with a first graphic 301 comprising a graphic 301A and a graphic 301B. Also, the second backsheet 222 is provided with a second graphic 302 comprising a graphic 302A and a graphic 302B. Herein, the graphic 301A of the first backsheet 221 is identical with the graphic 302A of the second backsheet 222. Similarly, the graphic 301B of the first backsheet 221 is identical with the graphic 302B of the second backsheet 222. Referring to the backsheet 221, the graphic 301A comprises a first longitudinal component 561A and a first transverse component 581A. Also, the graphic 301B comprises a second longitudinal component 561B and a second transverse component 581B. Both the first transverse component 581A and the second transverse component 581B extend substantially continuously from the first longitudinal edge 42 to the second longitudinal edge 44. In the manufacturing process, the backsheet web 100 is conveyed in the machine direction MD corresponding to the longitudinal direction L. The cross machine direction CD is perpendicular to the machine direction MD. The backsheet 22 is provided with a registration mark 34 on the second surface 38. Each pattern of the graphic 30 is the same throughout its entire length of the backsheet web 100. As the graphic 30 and the registration mark 34 are located on the opposite surfaces to each other, the detection of the registration mark is carried out without detection error in the manufacturing process.

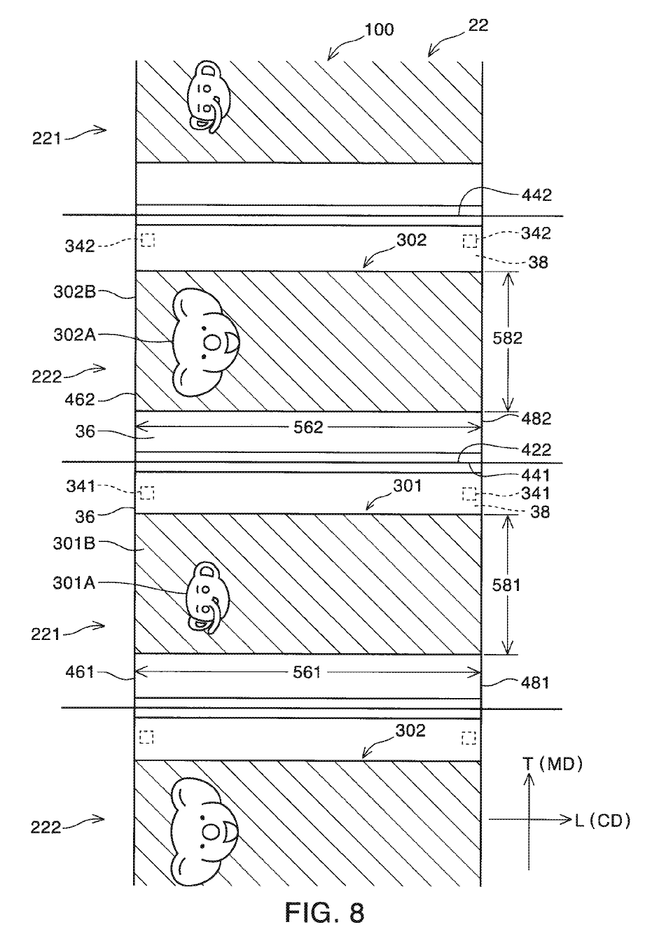

FIG. 8 illustrates another schematic diagram showing a portion of a continuous backsheet web 100 having consecutively spaced graphics and registration mark printed thereon. For the purpose of illustration, the ratio of the length and the width of the backsheet is modified. The backsheet web 100 of FIG. 8 is a status before being attached to the other components (e.g., topsheet, absorbent core, and the like). The backsheet web 100 consists of a series of backsheet 22 to be severed when the absorbent article is assembled. The series of backsheet 22 is provided with a graphic 30 respectively. For example, the backsheet web 100 comprises a series of a first backsheet 221 and a second backsheet 222. The first backsheet 221 comprises a first longitudinal edge 421 and a second longitudinal edge 441, and a first transverse edge 461 and a second transverse edge 481. Also, the second backsheet 222 comprises a first longitudinal edge 422 and a second longitudinal edge 442, and a first transverse edge 462 and a second transverse edge 482. The first backsheet 221 is provided with a graphic 301 and the backsheet 222 is provided with a graphic 302. The graphic 301 comprises a graphic 301A of an elephant and a graphic 301B of a colored pattern (the graphic 301A and the graphic 301B are collectively referred to as “the graphic 301”). The graphic 301A is located against the graphic 301B. The graphic 302 comprises a graphic 302A of a koala and a graphic 302B of a colored pattern (the graphic 302A and the graphic 302B are collectively referred to as “the graphic 302”). The graphic 302A is located against the graphic 302B. Each pattern of the graphic 301 and the graphic 302 is disposed alternately along the backsheet web 100. The graphic 301 comprises a longitudinal component 561 and a transverse component 581. The graphic 302 comprises a longitudinal component 562 and a transverse component 582. Referring to the backsheet 221, the longitudinal component 561 extends substantially continuously in the longitudinal direction L from the first transverse edge 461 to the second transverse edge 481. Also referring to the backsheet 222, the longitudinal component 562 extends substantially continuously in the longitudinal direction L from the first transverse edge 462 to the second transverse edge 482. In the manufacturing process, the backsheet web 100 is conveyed in the machine direction MD, which corresponds to the transverse direction T of the backsheet 22. The cross machine direction CD perpendicular to the machine direction MD corresponds to the longitudinal direction L. The first backsheet 221 is provided with a registration mark 341 on the second surface 38. The backsheet 221 is provided with a registration mark 342 on the second surface 38.

As illustrated in FIG. 8, using the backsheet web 100 which comprises a plurality of kinds of graphics makes it possible to manufacture absorbent articles which have different patterns on the surface. Such absorbent articles may be contained in a single package. The variety of graphics in a single package enhances the consumer's acceptance. Similar to the embodiment of FIG. 2, if the first graphic 301, the second graphic 302 and the registration mark 34 are located on the same surface of the backsheet 22, the detector may mistakenly detect the first graphic 301 and/or the second graphic 302 as a registration mark. Furthermore, when the backsheet 22 comprises more variations of graphic and accordingly the graphic includes more colors, there is a possibility to cause more detection error because the detector may sometimes not be able to distinguish the registration mark from the graphic. In the present invention, as the graphic and the registration mark are provided on the opposite surfaces of the backsheet to each other, the manufacturer can choose arbitrary colors for the graphic 30 of the backsheet 22 regardless of the registration mark 34.

The following briefly explains the basic structure of the disposable diaper of the present invention.

The topsheet 24 may be positioned adjacent the inner surface of the absorbent core 26 and may be joined thereto and to the backsheet 22 by attachment means such as those well known in the art. Suitable attachment means are described with respect to joining the backsheet 22 to the absorbent core 26. In an embodiment of the present invention, the topsheet 24 and the backsheet 22 are joined directly to each other in the diaper periphery and are indirectly joined together by directly joining them to the absorbent core 26 by any suitable attachment means. The topsheet 24 may be compliant, soft feeling, and non-irritating to the wearer's skin. Furthermore, the topsheet 24 may be liquid pervious permitting liquids (e.g., urine) to readily penetrate through its thickness. A topsheet 24 may be manufactured from a wide range of materials such as woven and nonwoven materials; polymeric materials such as apertured formed thermoplastic films, apertured plastic films, and hydroformed thermoplastic films; porous foams; reticulated foams; reticulated thermoplastic films; and thermoplastic scrims. Woven and nonwoven materials can be comprised of natural fibers (e.g., wood or cotton fibers), synthetic fibers (e.g., polymeric fibers such as polyester, polypropylene or polyethylene fibers) or from a combination of natural and synthetic fibers. The topsheet 24 can be rendered hydrophilic by treating it with hydrophilic finishing oil or a surfactant. Methods for the treatment for the topsheet 24 include spraying the topsheet 24 material with surfactant and immersing the material into the surfactant. A more detailed discussion of such a treatment and hydrophilicity is contained in U.S. Pat. No. 4,988,344 entitled Absorbent Articles with Multiple Layer Absorbent Layers” issued to Reising, et al. on Jan. 29, 1991 and U.S. Pat. No. 4,988,345 entitled Absorbent Articles with Rapid Acquiring Absorbent Cores” issued to Reising on Jan. 29, 1991, each of which is incorporated by reference herein. Alternatively, the topsheet 24 may be a carded nonwoven material which is formed by fibers treated with hydrophilic finishing oil.

The backsheet 22 is that portion of the diaper 20 which is generally positioned away from the wearer's skin and which prevents the exudates absorbed and contained in the absorbent core 26 from wetting articles which contact the diaper 20 such as bedsheets and undergarments. The backsheet 22 may be impervious to liquids (e.g., urine) and may be manufactured from a thin plastic film, although other soft, flexible liquid impervious materials may also be used. (As used herein, the term “flexible” refers to materials which are compliant and will readily conform to the general shape and contours of the human body.) While the backsheet 22 may be impervious to liquids, the backsheet 22 may permit moisture to escape from the diaper 20. Such a backsheet is called a breathable backsheet. The backsheet 22 may be positioned adjacent the external surface of the absorbent core 26 and may be joined thereto by any suitable attachment means known in the art. For example, the backsheet 22 may be secured to the absorbent core 26 by a uniform continuous layer of adhesive, a patterned layer of adhesive, or an array of separate lines, spirals, or spots of adhesive. Alternatively, the attachment means may comprise heat bonds, pressure bonds, ultrasonic bonds, dynamic mechanical bonds, or any other suitable attachment means or combinations of these attachment means as are known in the art. Embodiments of the present invention are also contemplated wherein the absorbent core 26 is not joined to the backsheet 22, and/or the topsheet 24 in order to provide greater extensibility in the front waist region and the rear waist region.

The diaper 20 may further comprise a nonwoven 32 over the backsheet 22. The nonwoven 32 may be joined with at least a portion of the external surface 74 of the backsheet 22. Alternatively, the nonwoven 32 may include any materials joined to the backsheet 22 such as woven webs, foams, scrims, loose fibers, or any other material or combination of materials known in the art that will give the diaper a cloth-like look and/or feel and is at a minimum air permeable. The nonwoven 32 may cover all or substantially all of the backsheet 22, or may cover only discrete predetermined portions. The nonwoven 32 may provide the diaper 20 with a low cost landing zone capable of engaging the hooks of a hook and loop type fastener. Such a landing zone could be utilized as a portion of a primary fastening system or as a means for disposing of a soiled diaper. The nonwoven 32 may comprise natural fibers (e.g., cotton or wood fibers), or may comprise fibers of polyethylene, polypropylene, polyester, or any combination of such fibers. Further, the nonwoven 32 may be carded, spunbond, meltblown or air-through bonded or have any other characteristic or be manufactured in any manner known in the art. The nonwoven 32 may be comprised of sufficient thermoplastic material to allow for thermal bonding of the material to other components of the diaper. A nonwoven may be a carded nonwoven made of 100% polypropylene fibers such as Sawabond 4111 manufactured by Miesstofwerk Christian Heinrich Sandler GmbH & Co. KG, Germany.

The backsheet film may comprise any known material. The backsheet film may be moisture pervious and/or liquid impervious. For example, the backsheet film may comprise a breathable microporous film composed of a thermoplastic resin and inorganic fillers dispersed in the thermoplastic resin. Suitable thermoplastic polymers include polyolefins such as polyethylenes, including liner low density polyethylene (LLDPE), low density polyethylene (LDPE), ultra low density polyethylene (ULDPE), high density polyethylene (HIDPE), or polypropylene and blends thereof with the above and other materials. Examples of other suitable thermoplastic polymers which may also be used include, but are not limited to, polyester, polyurethanes, compostable or biodegradable polymers, thermoplastic elastomers, and metallocene catalyst-based polymers (e.g., INSITEO available from Dow Chemical Company and ExxactO available from Exxon). The inorganic material or filler is selected from the group consisting of calcium carbonate, clay and titanium dioxide, with the preferred inorganic filler being calcium carbonate.

The absorbent core 26 may be any absorbent member which is generally compressible, conformable, non-irritating to the wearer's skin, and capable of absorbing and retaining liquids such as urine and other certain body exudates. The absorbent core 26 may be manufactured in a wide variety of sizes and shapes (e.g., rectangular, hourglass, T-shaped, asymmetric, etc.) and from a wide variety of liquid-absorbent materials commonly used in disposable diapers and other absorbent articles such as comminuted wood pulp which is generally referred to as airfelt. Examples of other suitable absorbent materials include creped cellulose wadding; chemically stiffened, modified or cross-linked cellulosic fibers; tissue including tissue wraps and tissue laminates; absorbent foams; absorbent sponges; superabsorbent polymers; absorbent gelling materials; or any equivalent material or combinations of materials. The configuration and construction of the absorbent core 26 may vary (e.g., the absorbent core may have varying caliper zones, a hydrophilic gradient, a superabsorbent gradient, or Jower average density and lower average basis weight acquisition zones; or may comprise one or more layers or structures). Furthermore, the size and absorbent capacity of the absorbent core 26 may also be varied to accommodate wearers ranging from infants through adults. However, the total absorbent capacity of the absorbent core 26 should be compatible with the design loading and the intended use of the diaper 20.

It is understood that the examples and embodiments described herein are for illustrative purpose only and that various modifications or changes will be suggested to one skilled in the art without departing from the scope of the present invention.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An absorbent article comprising a backsheet, a topsheet joined with the backsheet, and an absorbent core between the backsheet and the topsheet,
the backsheet extending in a first direction and in a second direction perpendicular to the first direction, and having a first surface, a second surface opposing to the first surface, a first edge and a second edge each extending substantially in the first direction, and a third edge and a fourth edge each extending substantially in the second direction,
the backsheet being provided with a graphic and a registration mark for adjusting the graphic to a predetermined position in the absorbent article, the registration mark being detected when the absorbent article is conveyed in the first direction in the manufacturing process,
the graphic being provided on the first surface, the graphic having a first direction component and a second direction component,
the second direction component extending substantially continuously from the first edge to the second edge, and
the registration mark being provided on the second surface.

2. The absorbent article of claim 1, the backsheet extending in a longitudinal direction and in a transverse direction perpendicular to the longitudinal direction, the backsheet having a first longitudinal edge, a second longitudinal edge, a first transverse edge and a second transverse edge,
the first direction being the longitudinal direction and the second direction being the transverse direction,
the first edge being the first longitudinal edge, the second edge being the second longitudinal edge, the third edge being the first transverse edge and the fourth edge being the second transverse edge.

3. The absorbent article of claim 1, the backsheet extending in a longitudinal direction and in a transverse direction perpendicular to the longitudinal direction, the backsheet having a first longitudinal edge, a second longitudinal edge, a first transverse edge and a second transverse edge,
the first direction being the transverse direction and the second direction being the longitudinal direction,
the first edge being the first transverse edge, the second edge being the second transverse edge, the third edge being the first longitudinal edge and the fourth edge being the second longitudinal edge.

4. The absorbent article of claim 1, the backsheet being provided with a single graphic, wherein the second direction component of the single graphic extends substantially continuously from the first edge to the second edge.

5. The absorbent article of claim 1, the backsheet being provided with a plurality of graphics, each of the graphics having a first direction component and a second direction component, each of the second direction components extending only in a portion between the first edge and the second edge,
the plurality of the second direction components forming a composite second direction component,
wherein the composite second direction component extends substantially continuously from the first edge to the second edge.

6. The absorbent article of claim 1, the first surface being an external surface of the backsheet and the second surface being a body-facing surface of the backsheet.

7. The absorbent article of claim 6, the backsheet having a thickness direction perpendicular to the first direction and the second direction,
the graphic on the first surface partially covering the registration mark on the second surface in the thickness direction.

8. The absorbent article of claim 6, the graphic on the first surface covering the entirety of the registration mark on the second surface in the thickness direction.

9. The absorbent article of claim 6, the registration mark being substantially unperceivable from the external side of the absorbent article.

10. The absorbent article of claim 6, the registration mark being substantially perceivable from the external side of the absorbent article.
